(19) United States
(12) Patent Application Publication
(10) Pub. No.: US 2010/0152589 A1
(43) Pub. Date: Jun. 17, 2010

(54) PROBE CABLE ACCOMMODATION APPARATUS AND ULTRASOUND DIAGNOSTIC SYSTEM

(76) Inventors: Akimasa Asai, Tokyo (JP); Kazuhiko Hayakawa, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/635,868

(22) Filed: Dec. 11, 2009

(30) Foreign Application Priority Data
Dec. 12, 2008 (JP) 2008-316791

(51) Int. Cl. A61B 8/14

(52) U.S. Cl. 600/459

(57) ABSTRACT

A probe cable accommodation apparatus includes a receptacle housing accommodating a plurality of probe cables. Each of the probe cables is connected at one end thereof to an ultrasound diagnostic system and has at an opposite end thereof an ultrasound probe. The interior of the receptacle housing is partitioned by a partition plate to form a plurality of receptacle chambers. The probe cables are accommodated in the receptacle chambers respectively.

### CROSS REFERENCE TO RELATED APPLICATIONS

This application claims the benefit of Japanese Patent Application No. 2008-316791 filed Dec. 12, 2008, which is hereby incorporated by reference in its entirety.

### BACKGROUND OF THE INVENTION

The embodiments described herein relate to a probe cable accommodation apparatus for accommodating probe cables, as well as an ultrasound diagnostic system.

An ultrasound diagnostic system that generates an ultrasound image on the basis of an echo signal obtained by transmitting an ultrasound wave to a subject is provided with an ultrasound probe for transmission and reception of an ultrasound wave, the probe being connected to the ultrasound diagnostic system through a probe cable (see, for example, Japanese Unexamined Patent Publication No. 2003-339708).

In the conventional ultrasound diagnostic system, the probe cable is disposed as it is in the exterior of the system and is therefore an obstacle when the ultrasound probe is not in use; besides, the whole of the ultrasound diagnostic system is bad to look at.

In this connection the applicant of the present application has made a study about a probe cable accommodation apparatus capable of accommodating the probe cable. As a result there occurred the following problem. In an ultrasound diagnostic system to which a plurality of ultrasound probes can be connected, plural probe cables are used, and when an attempt is made to accommodate those plural probe cables, the cables get entangled each other and the operator feels the cable accommodation operation troublesome.

### BRIEF DESCRIPTION OF THE INVENTION

In a first aspect of the invention there is provided a probe cable accommodation apparatus including a receptacle housing accommodating a plurality of probe cables, the probe cables being each connected at one end thereof to an ultrasound diagnostic system and having at an opposite end thereof an ultrasound probe, the interior of the receptacle housing being partitioned by a partition plate to form a plurality of receptacle chambers, the probe cables being accommodated in the receptacle chambers respectively.

In a second aspect of the invention there is provided, in combination with the above first aspect, a probe cable accommodation apparatus wherein the receptacle housing has openings for the probe cables accommodated in the receptacle chambers to get in and out, and the probe cables are received in U shape from the openings into the receptacle chambers respectively.

In a third aspect of the invention there is provided, in combination with the above first or second aspect, a probe cable accommodation apparatus wherein rollers for abutment thereagainst of each of the probe cables are provided in each of the openings.

In a fourth aspect of the invention there is provided, in combination with the above third aspect, a probe cable accommodation apparatus further including as the rollers a first roller and a second roller both sandwiching the probe cable therebetween, the first and second rollers being rotatable as the probe cable gets in or out of the associated receptacle chamber and mutually pressing the probe cable to prevent the probe cable from getting and out of the receptacle chamber.

In a fifth aspect of the invention there is provided, in combination with any of the above first to third aspects, a probe cable accommodation apparatus wherein a cable stopper for preventing each of the probe cables from getting in and out of the associated receptacle chamber is provided in each of the openings.

In a sixth aspect of the invention there is provided, in combination with any of the above second to fifth aspects, a probe cable accommodation apparatus wherein the receptacle chambers are each formed in a tubular shape having a parallelogrammic or rhombic section.

In a seventh aspect of the invention there is provided, in combination with any of the above second to sixth aspects, a probe cable accommodation apparatus wherein the probe cables are received in U shape from the openings toward bottoms of the receptacle chambers respectively and a movable pulley is engaged on a lower end of each of the U-shaped probe cables.

In an eighth aspect of the invention there is provided, in combination with the above seventh aspect, a probe cable accommodation apparatus wherein the movable pulley is urged toward the bottom by a weight.

In a ninth aspect of the invention there is provided, in combination with the above seventh or eighth aspect, a probe cable accommodation apparatus wherein the movable pulley is held vertically movably by a rail.

In a tenth aspect of the invention there is provided, in combination with any of the above seventh to ninth aspects, a probe cable accommodation apparatus wherein the movable pulley is supported by an elastic member fixed to the bottom of the associated receptacle chamber.

In an eleventh aspect of the invention there is provided, in combination with the above tenth aspect, a probe cable accommodation apparatus wherein the elastic member is a spring.

In a twelfth aspect of the invention there is provided, in combination with any of the above second to sixth aspects, a probe cable accommodation apparatus wherein the probe cables are received in U shape from the openings toward bottoms of the receptacle chambers respectively, and a weight is engaged on a lower end of each of the U-shaped probe cables in a slidable state with respect to the probe cable.

In a thirteenth aspect of the invention there is provided an ultrasound diagnostic system including the probe cable accommodation apparatus described in any of the above first to twelfth aspects.

In a fourteenth aspect of the invention there is provided, in combination with the above thirteenth aspect, an ultrasound diagnostic system further including, in addition to the probe cable accommodation apparatus, an operating device for operator's input of instructions, an ultrasound probe for ultrasound wave transmission and reception, an image display device for display of an ultrasound image, and a processing device for making control to generate an ultrasound image on the basis of an echo signal obtained by activating the ultrasound probe and display the ultrasound image on the image display device, the operating device being separate from all of the probe cable accommodation apparatus, the image display device and the processing device and disposed on the side opposite to the processing device with a subject-carrying table therebetween.

In a fifteenth aspect of the invention there is provided, in combination with the above fourteenth aspect, an ultrasound diagnostic system further including a mounting section for mounting the image display device and wherein the image display device is mounted to the mounting section through an arm having a movable portion for changing the position of the image display device.

According to the first aspect, since the interior of the receptacle housing for accommodating a plurality of the probe cables is partitioned by the partition plate to form plural receptacle chambers and the probe cables are accommodated in the receptacle chambers respectively, the plural probe cables can be accommodated without mutual entanglement.

According to the second aspect the probe cables accommodated in U shape within the receptacle chambers can be allowed to get in and out of the openings.

According to the third aspect, with the rollers, the probe cables can be allowed to get in and out of the receptacle chambers smoothly.

According to the fourth aspect, with the first and second rollers, not only the probe cables can be allowed to get in and out of the receptacle chambers smoothly, but also the probe cables can be prevented from going out of the openings or being drawn into the receptacle chambers against the operator's will.

According to the fifth aspect, with the cable stoppers, the probe cables can be prevented from going out of the openings or being drawn into the receptacle chambers against the operator's will.

According to the sixth aspect, since the probe cables are accommodated in U shape respectively into receptacle chambers formed in a tubular shape of a parallelogrammic or rhombic section, the probe cables can be accommodated in a state in which the radius of curvature of a lower end portion of each of the U-shaped probe cables is large, for example in comparison with a case where the receptacle chambers are formed in a tubular shape of a rectangular section. Therefore, as compared with the case where the receptacle chambers are each formed in a tubular shape of a rectangular section, the probe cables can be accommodated at a large radius of curvature and without forced bending, even if the thickness of the receptacle housing is made small. Thus, the receptacle housing can be made thin.

According to the seventh aspect, with the movable pulley engaged on the lower end of each of the probe cables, it is possible to restrict the radius of curvature of the lower end portion of each of the U-shaped probe cables accommodated in the receptacle chambers. Consequently, it is possible to thin the receptacle housing.

According to the eighth aspect, the weight permits the probe cables to be drawn into the receptacle chambers easily.

According to ninth aspect, since the movable pulley is held vertically movably by the rail, it is possible to stabilize the vertical movement of the movable pulley.

According to the tenth aspect, since the probe cables are each urged toward the bottom by the elastic member, they can be drawn into the receptacle chambers easily.

According to the eleventh aspect, the probe cables drawn out from the receptacle chambers can each be urged toward the bottom by the spring.

According to the twelfth aspect, since the probe cables are each urged toward the bottom by the weight, they can be drawn into the receptacle chambers easily.

According to the thirteenth aspect it is possible to obtain an ultrasound diagnostic system having the effects of the inventions in the first to twelfth aspects.

According to the fourteenth aspect, the operating device is disposed on the operator side with respect to the table, and the probe cable accommodation apparatus, the image display device and the processing device are disposed on the side opposite to the operating device with the table therebetween. Therefore, as compared with the conventional ultrasound diagnostic system in which the operating device, the image display device and the processing device are integral, the operator-side space becomes wider and it is possible to effectively utilize the space of the room where the ultrasound diagnostic system is installed.

According to the fifteenth aspect, since the image display device is mounted through the arm having the movable portion, the image display device can be disposed at a position where it can be seen easily by the operator.

### BRIEF DESCRIPTION OF THE DRAWINGS

FIG. 1 is a front view showing a probe cable accommodation apparatus and an ultrasound diagnostic system according to an embodiment of the invention.

FIG. 2 is a partially cutaway right side view of the probe cable accommodation apparatus and the ultrasound diagnostic system both shown in FIG. 1.

FIG. 3 is a plan view of the probe cable accommodation apparatus and the ultrasound diagnostic system both shown in FIG. 1.

FIG. 4 is a sectional view taken on line A-A in FIG. 1.

FIG. 5 is a partially cutaway, enlarged front view of openings and the vicinity thereof in the probe cable accommodation apparatus and the ultrasound diagnostic system both shown in FIG. 1.

FIG. 6 is a partially cutaway, enlarged side view of an opening and the vicinity thereof in the probe cable accommodation apparatus and the ultrasound diagnostic system both shown in FIG. 1.

FIG. 7 is a partially enlarged plan view of a probe cable accommodation apparatus in an ultrasound diagnostic system according to a modification of the first embodiment.

FIG. 8 is a partially cutaway side view of a probe cable accommodation apparatus and an ultrasound diagnostic system according to a second embodiment of the invention.

FIG. 9 is a front view of the probe cable accommodation apparatus and the ultrasound diagnostic system both shown in FIG. 8.

FIG. 10 is an enlarged side view of a movable pulley and the vicinity thereof in an exposed state of the interior by downwardly sliding a cover attached to a receptacle housing of the probe cable accommodation apparatus according to the second embodiment.

FIG. 11 is an enlarged side view of a movable pulley lock and the vicinity thereof in a released state of the movable pulley lock.

FIG. 12 is an enlarged side view of the movable pulley lock and the vicinity thereof in an operating state of the movable pulley lock.

FIG. 13 is an enlarged side view of the movable pulley and the vicinity thereof in a released state of the cable lock.

FIG. 14 is a partially enlarged sectional view of a cable accommodation apparatus according to a modification of the second embodiment.

### DETAILED DESCRIPTION OF THE INVENTION

Embodiments of the invention will be described below in detail with reference to the drawings.

### First Embodiment

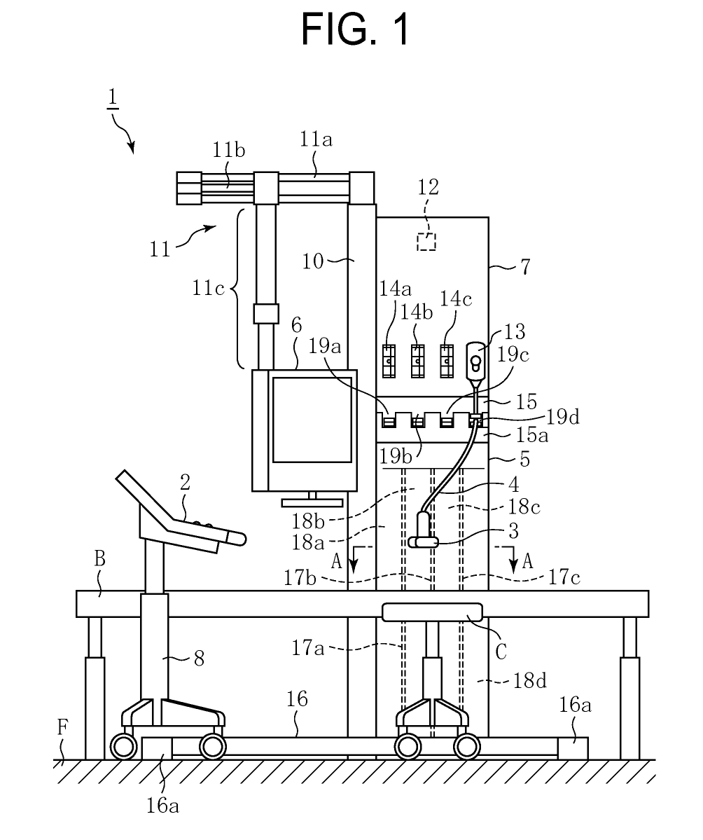

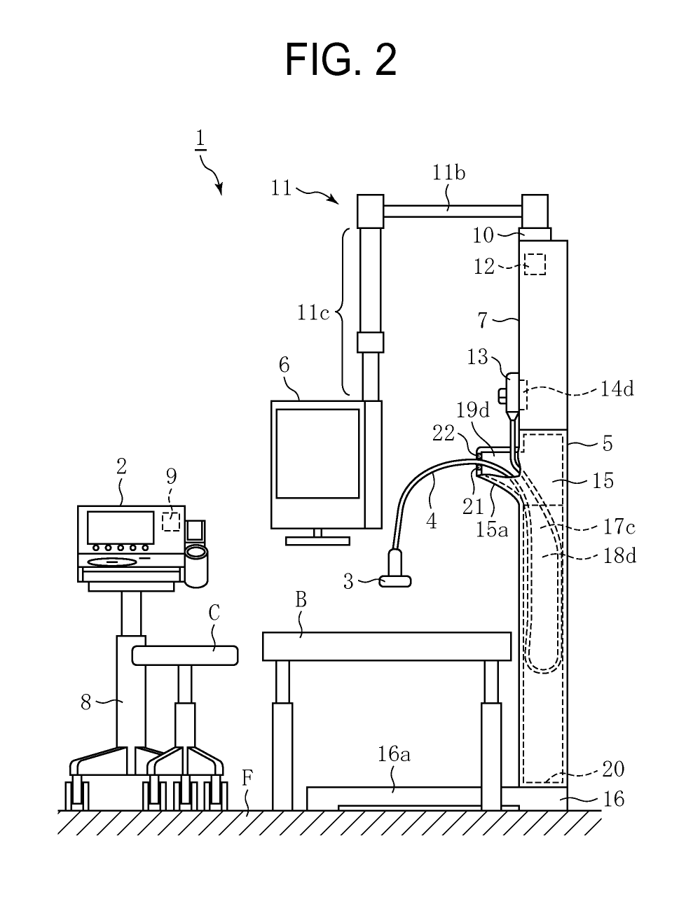

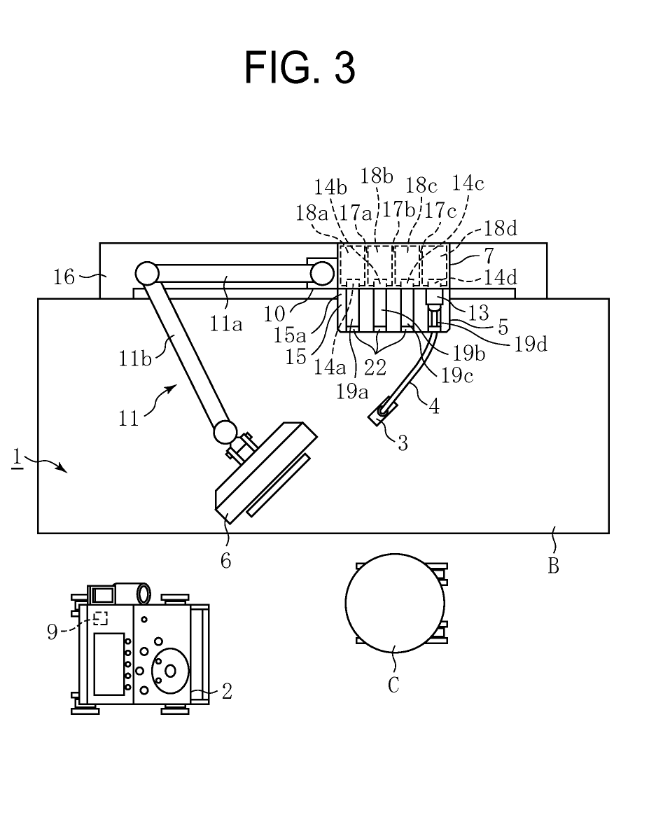

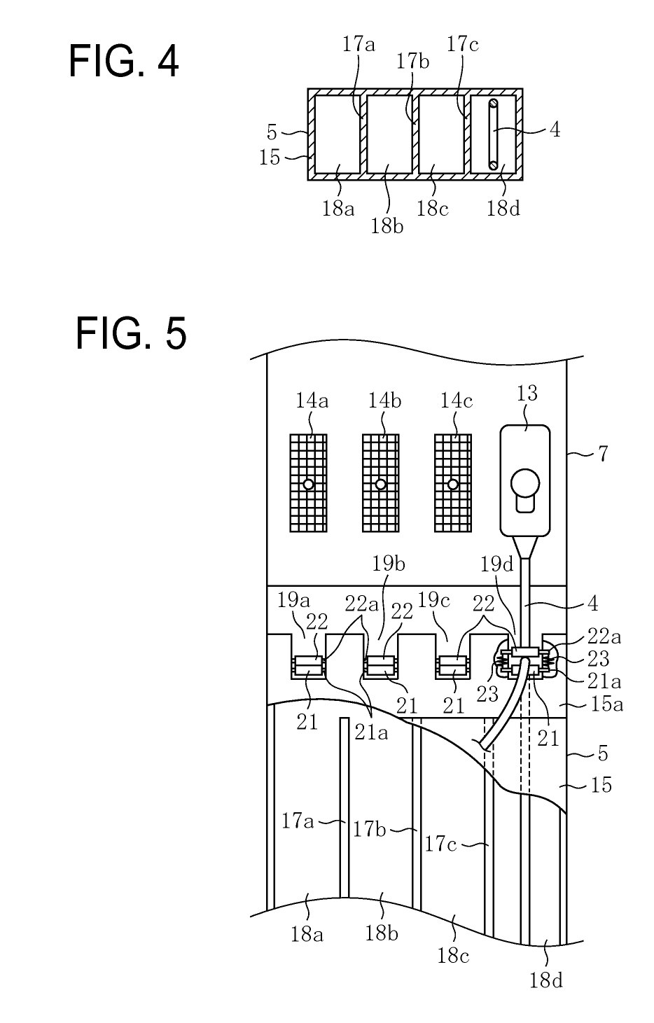

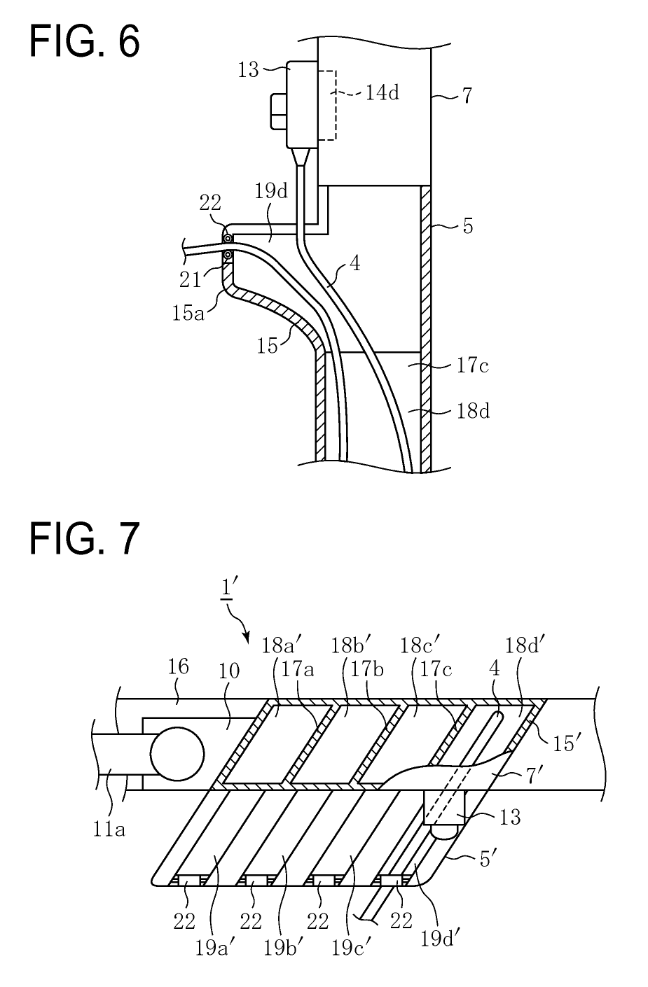

A first embodiment of the invention will first be described. FIG. 1 is a front view showing a probe cable accommodation apparatus and an ultrasound diagnostic system according to a first embodiment of the invention, FIG. 2 is a partially cutaway right side view of the probe cable accommodation apparatus and the ultrasound diagnostic system both shown in FIG. 1, FIG. 3 is a plan view of the probe cable accommodation apparatus and the ultrasound diagnostic system both shown in FIG. 1, FIG. 4 is a sectional view taken on ling A-A in FIG. 1, FIG. 5 is a partially cutaway, enlarged front view of openings and the vicinity thereof in the probe cable accommodation apparatus and the ultrasound diagnostic system both shown in FIG. 1, and FIG. 6 is a partially cutaway, enlarged side view of an opening and the vicinity thereof in the probe cable accommodation apparatus and the ultrasound diagnostic system both shown in FIG. 1.

An ultrasound diagnostic system 1 includes an operating device 2 for operator's input of instructions, an ultrasound probe 3 for ultrasound wave transmission and reception, a probe cable accommodation apparatus 5 for accommodating a probe cable 4 of the probe 3, an image display device 6 for display of an ultrasound image, and a processing device 7 for making control to generate an ultrasound image on the basis of an echo signal obtained by activating the ultrasound probe 3 in accordance with an instruction of the operator and display the ultrasound image on the image display device 6.

The operating device 2 is separate from all of the probe cable accommodation apparatus 5, the image display device 6 and the processing device 7 and is installed on a carrier 8 with caster so that the position thereof can be changed freely. With respect to a bed B on which a subject lies down, the operating device 2 is positioned on the side where a chair C for the operator is placed. The bed B is an example of the table defined in the invention. The operating device 2 has an operating device-side radio communication section 9 for radio communication with the processing device 7. When the operator inputs an instruction through the operating device 2, that instruction signal is transmitted to the processing device 7 by the operating device-side radio communication section 9.

The image display device 6 is mounted through an arm 11 to a support post 10 that is fixed to side faces of both probe cable accommodation apparatus 5 and processing device 7. The support post 10 is an example of the mounting section defined in the invention. Though not shown, the image display device 6 may be mounted to the processing device 7 through an arm. Or, though not shown, the image display device 6 may be mounted through an arm to the ceiling or wall of the room in which the ultrasound diagnostic system is installed.

The arm 11 is made up of a first horizontal arm 11a extending from the support post 10, a second horizontal arm 11b extending horizontally from the first arm 11a, and a vertical arm 11c extending perpendicularly from the second horizontal arm 11b. A cable (not shown) is disposed within the arm 11 and data of the ultrasound image generated in the processing unit 7 are inputted to the image display device 6 through the cable.

The first horizontal arm 11a is adapted to rotate horizontally at the connection thereof with the support post 10. The second horizontal arm 11b is adapted to rotate horizontally at the connection thereof with the first horizontal arm 11a. Further, the vertical arm 11c, whose length is variable, is adapted to rotate horizontally at the connection thereof with the second horizontal arm 11b. Since the arm 11 has such movable portions, the image display device 6 can be moved according to the attitude of an inspector to a position where it is easy to see, thus permitting the inspector to make inspection at a natural attitude.

The image display device 6 is adapted to rotate vertically at the connection thereof with the vertical arm 11c, thus making it possible to adjust the screen angle.

The processing device 7 has a processing device-side radio communication section 12 for radio communication with the operating device 2. Moreover, the processing device 7 has probe connectors 14a, 14b, 14c and 14d for connection thereto of a connector 13 of the probe cable 4 which connects the ultrasound probe 3 and the processing device 7 with each other. In this embodiment, the connector 13 is connected to only the probe connector 14d.

The processing device 7 and the cable accommodation apparatus 5 are integral with each other and are disposed on the side opposite to the operating device 2 side with respect to the bed B. The probe cable accommodation apparatus 5 includes a receptacle housing 15 for accommodating the probe cable 4. The receptacle housing 15 is mounted on a base 16 that is installed on a floor surface F. The base 16 is provided at both ends thereof with a pair of tipping preventing legs 16a and is U-shaped in plan. With the tipping preventing legs 16a, it is possible to prevent tipping of the cable accommodation apparatus 5, as well as the image display device 6 and the processing device 7 which are integral with the cable accommodation apparatus 5. The base 16 may be fixed to the floor surface F with anchor bolts or the like though not specially shown.

The receptacle housing 15 has an upper projecting portion 15a and is formed in an inverted L shape in side view. The interior of the receptacle housing 15 is partitioned with three partition plates 17a, 17b and 17c into four receptacle chambers 18a, 18b, 18c and 18d. The receptacle chambers 18a-18d are each formed in a tubular shape of a rectangular section to accommodate probe cables 4 respectively. Thus, the receptacle housing 15 can accommodate a total of four probe cables. In the illustrated example there is shown one probe cable 4 accommodated in only the receptacle chamber 18d.

Four openings 19a, 19b, 19c and 19d are formed in the projecting portion 15a of the cable receptacle housing 15. Probe cables get in and out of the receptacle chambers 18a-18d through the openings 19a-19d. The openings 19a-19d are positioned at a height of approximately 120.0 centimeters (cM) or more from the floor surface F. With the openings 19a-19d formed at such a position of approximately 120.0 cm or more in height from the floor surface F, probe cables 4 get in or out of the receptacle chambers from a position sufficiently higher than the patient lying down on the bed B. Thus, it is possible to prevent probe cables 4 from giving an unpleasant feeling to the patient.

The probe connectors 14a-14d are positioned higher than the openings 19a-19d and probe cables 4 connected to the probe connectors 14a-14d are accommodated in the receptacle chambers 18a-18d through the openings 19a-19d respectively. More specifically, probe cables 4 (the illustrated probe cable 4) are accommodated in U shape from the openings 19a-19d toward a bottom 20 of the receptacle housing 15 (the bottom of the receptacle chamber 18d).

A first roller 21 and a second roller 22 are provided in each of the openings 19a-19d so as to sandwich the illustrated probe cable 4 in between the rollers. The first and second rollers 21, 22 are provided in a number corresponding to the number of probe cables capable of being accommodated in the probe cable accommodation apparatus 5. In this embodiment there are provided four pairs of first rollers 21 and second rollers 22.

A further description will now be given about the first and second rollers 21, 22. The first and second rollers 21, 22 are adapted to rotate as probe cables 4 get in or out of the receptacle chambers 18a-18d, whereby probe cables 4 can get in and out of the receptacle chambers 18a-18d smoothly. As shown in FIG. 5, shafts 21a and 22a of the first and second rollers 21 are connected together by tension springs 23 (only the first and second rollers 21, 22 disposed in the opening 19d are shown). Consequently, the first and second rollers 21, 22 mutually urge the probe cable 4 sandwiched in between the rollers to prevent the probe cable from getting in or out with respect to the receptacle chambers 18a-18d. Thus, with the first and second rollers 21, 22, probe cables 4 can be prevented from getting out of the openings 19a-19d or being drawn into the receptacle chambers 18a-18d against the operator's will. The first and second rollers 21, 22 are an example of the rollers and cable stopper defined in the invention. However, the rollers and cable stopper defined in the invention are not limited to the above construction. For example, slits capable of grippingly holding probe cables 4 in an elastically deformed state may be formed in the openings 19a-19d to constitute cable stoppers.

Each of the second rollers 22 is movable vertically, and when the pressure imposed on the illustrated probe cable 4 by the associated first and second rollers 21, 22 is relieved by moving the associated second roller 22 vertically against the resilience of the tension spring 23, it becomes possible to let the probe cable 4 get in or out of the receptacle chamber 18d smoothly. However, a modification may be made such that the pressure imposed on the probe cable 4 by the first and second rollers 21, 22 is relieved by moving not the second roller 22 but the first roller 21 downward. Further, for relieving the pressure on the probe cable 4 by the first roller 21 or the second roller 22, there may be used a button connected to the first or the second roller mechanically to move the roller or there may be used a motor for moving the first or the second roller.

In the cutaway portion of FIG. 5, as to the structure for mounting the shafts 21a and 22a and as to the structure for mounting the tension springs 23 to the shafts 21a and 22a, though the details are not shown, the shafts 21a and 22a are mounted in a rotatable state and the tension springs 23 are mounted to the shafts 21a and 22a so as to permit rotation of those shafts.

According to the first embodiment described above, since probe cables 4 are accommodated in the receptacle chambers 18a-18d respectively, it is possible to accommodate plural probe cables without mutual entanglement.

According to the above embodiment, moreover, the operating device 2 is separate from all of the probe cable accommodation apparatus 5, the image display device 6 and the processing device 7, and the probe cable accommodation apparatus 5, the image display device 6 and the processing device 7 are disposed on the side opposite to the operating device 2 with the bed B therebetween. With such a construction, the operator-side space becomes wider than in the prior art and it is possible to effectively utilize the space of the room where the ultrasound diagnostic system 1 is installed, as compared with the conventional ultrasound diagnostic system wherein the operating device, image display device and processing device are integral with one another.

Moreover, since the image display device 6 is supported by the arm 11 extending from the support post 10, the operator-side space with respect to the bed B is not occupied by a supporting structure for the image display device 6. Further, with the arm 11, the image display device 6 can be moved to a position where the operator is easy to see the display device, so that the operator can see the image display device 6 at an easy attitude at the time of inspection or the like.

Next, a modification of the first embodiment will be described below with reference to FIG. 7. FIG. 7 is a partially enlarged plan view of a probe cable accommodation apparatus and the vicinity thereof in an ultrasound diagnostic system according to a modification of the first embodiment.

In a probe cable accommodation apparatus 5′ in an ultrasound diagnostic system 1′ according to this modification, a receptacle housing 15′ is formed in the shape of a parallelogram in plan and receptacle chambers 18a′, 18b′, 18c′ and 18d′ are each formed in a tubular shape having a parallelogrammic section. A processing device 7′, which is integral with the probe cable accommodation apparatus 5′ is also formed in the shape of a parallelogram in plan. Further, openings 19a′, 19b′, 19c′ and 19d′ are also formed in a parallelogrammic shape in plan. However, the receptacle chambers 18a′-18d′ may be rhombic in section and in this case the receptacle housing 15′, the processing device 7′ and the openings 19a′-19d′ may also be formed in a rhombic shape in section.

According to this modification, by accommodating probe cables 4 in U shape into the receptacle chambers 18a′-18d′ of a parallelogrammic section, the probe cables can each be accommodated in a state in which the radius of curvature of the cable lower end portion is larger than in the case of accommodating probe cables in the receptacle chambers 18a-18d of a rectangular section. The probe cables 4, with the rigidity thereof, have a certain extent when accommodated in U shape. Therefore, the thinner the receptacle housing 15, the more forcibly are bent the probe cables 4 and urged against the inner wall surfaces of the receptacle chambers 18a-18d. According to this modification, however, even if the receptacle housing 15′ is made thin, the probe cables 4 can be accommodated at a large radius of curvature without being bent forcibly, as compared with the receptacle housing 15 having the receptacle chambers 18a-18d of a rectangular section. Consequently, the probe cables 4 can be allowed to get in and out smoothly without being caught on the inner wall surfaces of the receptacle chambers 18a-18d. Thus, according to this modification, the receptacle housing 15′ can be made thin without impairing the smooth getting-in and -out motion of the probe cables 4 and hence the space of the installed room of the ultrasound diagnostic system 1 can be utilized more effectively.

### Second Embodiment

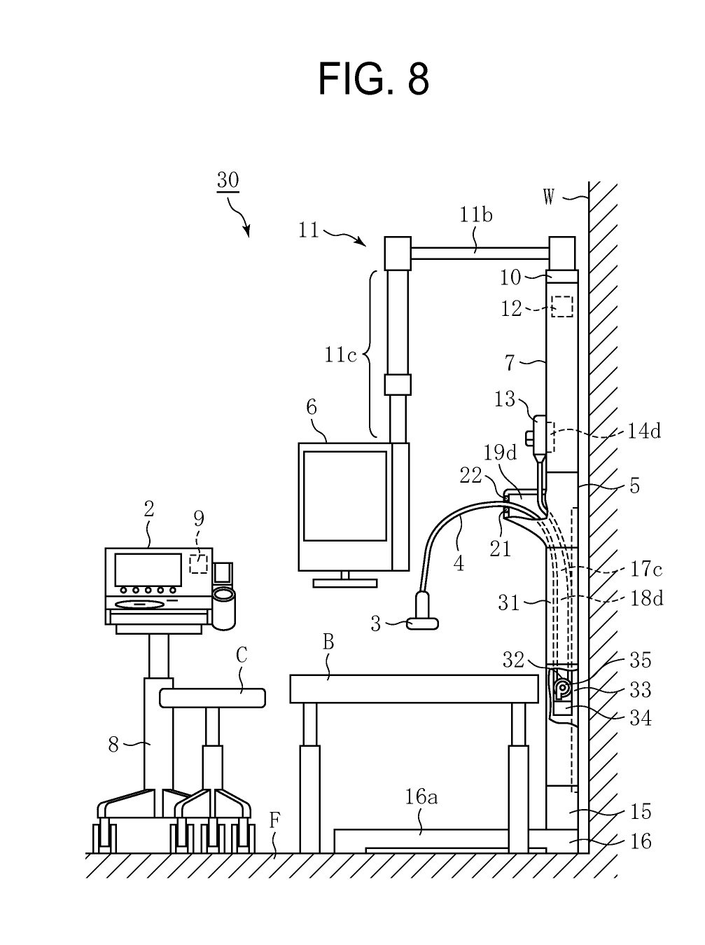

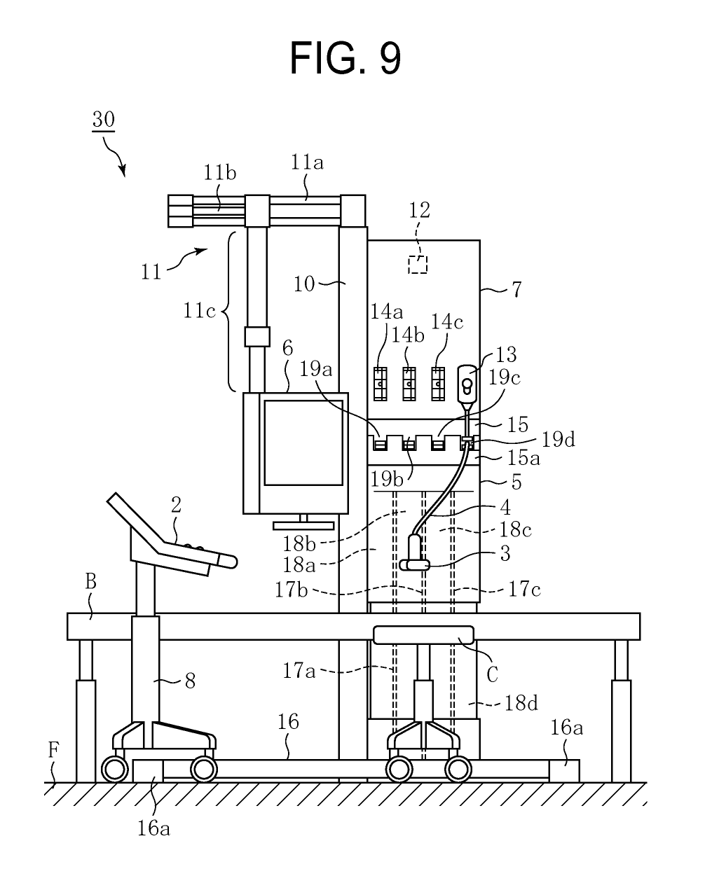

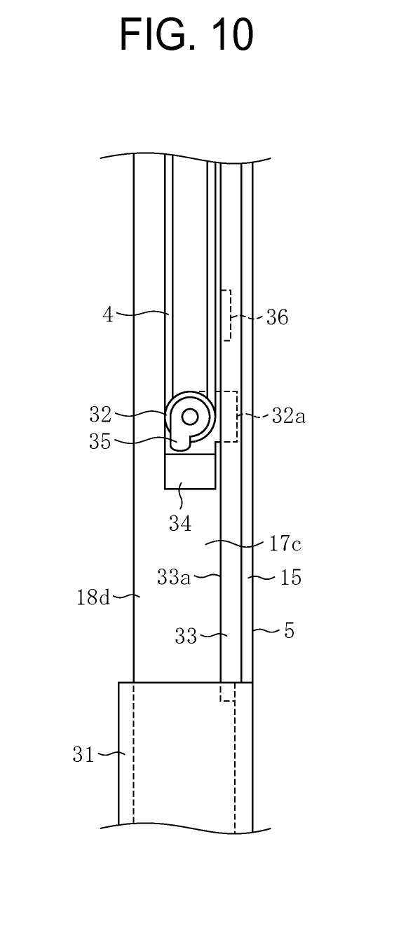

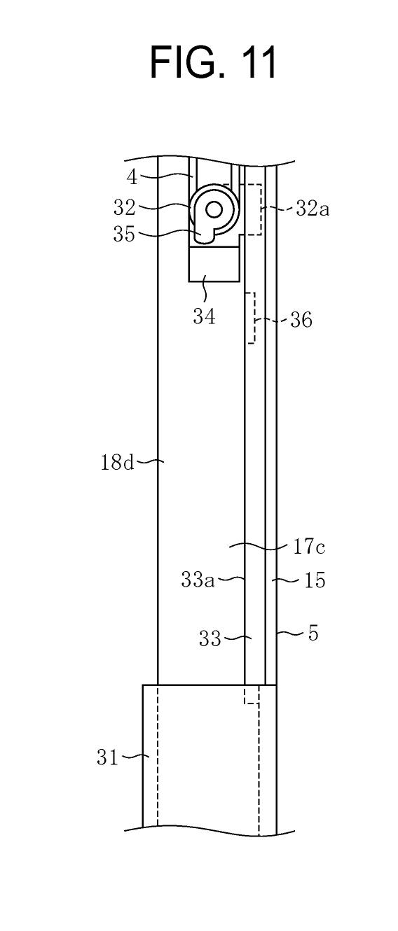

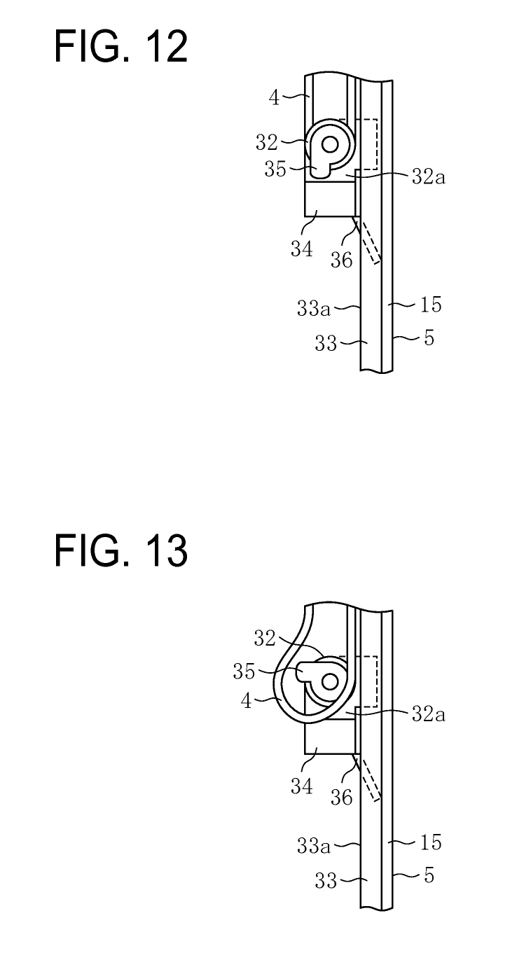

A second embodiment of the invention will be described below with reference to FIGS. 8 to 13. FIG. 8 is a partially cutaway side view showing a probe cable accommodation apparatus and an ultrasound diagnostic system according to a second embodiment of the invention, FIG. 9 is a front view of the probe cable accommodation apparatus and the ultrasound diagnostic system both shown in FIG. 8, FIG. 10 is an enlarged side view of a movable pulley and the vicinity thereof in an exposed state of the interior by downwardly sliding a cover attached to a receptacle housing of the probe cable accommodation apparatus according to the second embodiment, FIG. 11 is an enlarged side view of a movable pulley lock and the vicinity thereof in a released state of the movable pulley lock, FIG. 12 is an enlarged side view of the movable pulley lock and the vicinity thereof in an operating state of the movable pulley lock, and FIG. 13 is an enlarged side view of the movable pulley and the vicinity thereof in a released state of a cable lock.

The ultrasound diagnostic system 30 according to this second embodiment is the same in basic construction as the ultrasound diagnostic system 1 of the first embodiment, provided a vertically slidable cover 31 is provided on the receptacle housing 15 of the probe cable accommodation apparatus 5. The partition plates 17a-17c are in a separated state from the cover 31 so as to permit sliding of the cover 31. The receptacle chambers 18a-18d are partially exposed by sliding the cover 31.

Movable pulleys 32 are engaged respectively on lower ends of U-shaped probe cables 4 accommodated in the receptacle chambers 18a-18d of the probe cable accommodation apparatus 5. The movable pulleys 32 are held by rails 33 respectively so as to be vertically movable through holders 32a, the rails 33 being fixed vertically to a rear surface of the receptacle housing 15 within the receptacle chambers 18a-18d. The movable pulleys 32 move vertically in a stable manner because they are held by the rails 33.

With weights 34, the movable pulleys 32 are urged toward the bottom 20 (downward). The weights 34 act as a force for drawing probe cables 4 smoothly into the receptacle chambers 18a-18d. How heavy the weights 34 should be is set at a value which makes it possible to prevent the probe cables 4 from getting in and out of the receptacle chambers under the pressure imposed thereon by the first and second rollers 21, 22.

Further, a cable lock 35 is installed in each movable pulley 32. The cable lock 35 is constructed of a rotatable lever-like member. With the cable lock 35, each probe cable 4 is prevented from being disengaged from the movable pulley 32, whereby both stability and reliability are improved.

A movable pulley lock 36 is attached to each rail 32. The movable pulley lock 36 is a seesaw-like member adapted to move like a seesaw and thereby change the attitude. The movable pulley lock 36 is disposed at a position where it can be exposed when the cover 31 is slid downward. With the movable pulley lock 36, as will be described later, the movable pulley 32 with probe cable 4 disengaged therefrom can be allowed to stop at a predetermined position and thus it becomes easier to carry out the probe cable engaging and disengaging work for the movable pulley 32.

FIGS. 11 to 13 shows in what procedure the illustrated probe cable 4 is to be disengaged from the movable pulley 32. For disengaging the cable 4 from the movable pulley 32, first the cover 31 is slid downward, then in a partially exposed state of the receptacle chambers 18a-18d, the probe cable 4 is drawn out and the movable pulley 32 is raised to a position higher than the movable pulley lock 36, as shown in FIG. 11.

Next, as shown in FIG. 12, the movable pulley lock 36 is moved like a seesaw, allowing it to project beyond an upper surface 33a of the rail 33, and the movable pulley 32 is lowered and put on the movable pulley lock 36. Then, as shown in FIG. 13, the cable lock 35 is turned up and the probe cable 4 is disengaged from the movable pulley 32.

The procedure for engaging the movable pulley 32 on the probe cable 4 is reverse to the procedure for disengaging the probe cable 4 from the movable pulley 32.

According to this second embodiment described above, the radius of curvature of each of U-shaped probe cables accommodated in the receptacle chambers 18a-18d can be restricted by the movable pulleys 32. Consequently, as compared with the case where the movable pulleys 32 are not used, even if the receptacle chambers 18a-18d become narrower as a result of thinning the receptacle housing 15, probe cables 4 can be allowed to get in and out of the receptacle chambers 18a-18d smoothly without being caught on the inner wall surfaces of the receptacle chambers. Thus, it is possible to thin the receptacle housing 15 without impairing the smoothness in ingress and egress of the probe cables 4, so that when the probe cable accommodation apparatus 5 and the processing apparatus 7 are disposed near a wall W, it is possible to narrow the gap between the bed B and the wall W. As a result, the space on the operating device 2 side becomes wider and hence the space of the room where the ultrasound diagnostic system 1 is installed can be utilized more effectively.

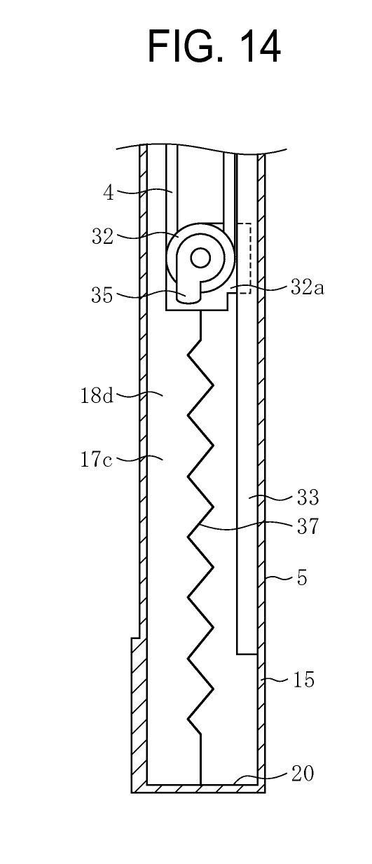

A modification of the second embodiment will be described below. FIG. 14 is a partially enlarged sectional view of a cable accommodation apparatus according to a modification of the second embodiment, provided the base 16 is not shown therein.

In this modification of the second embodiment the interior construction of the receptacle chambers 18a-18d is different from that in the first embodiment. The movable pulleys 32 disposed within the receptacle chambers 18a-18d (only the receptacle chamber 18d is shown in FIG. 14) are held by the rails 33 and are supported by springs 37 fixed to the bottom 20. The springs 37 are secured to the holders 32a. In this modification the weights 34 are not provided, but instead the springs 37 are provided. With the springs 37, the movable pulleys 32 are urged toward the bottom 20 (downward), whereby the illustrated probe cable 4 can be smoothly drawn into the receptacle chamber 18d. The springs 37 are an example of the elastic member defined in the invention.

The resilience of each of the springs 37 is set to the extent that the ingress and egress of the associated probe cable 4 can be stopped with the pressure imposed thereon by the first and second rollers 21, 22.

Although the invention has been described above by way of embodiments thereof, it goes without saying that various changes may be made within the scope not altering the gist of the invention. For example, though not specially shown, a weight, in place of the movable pulley 32, may be engaged on the lower end of the U-shaped probe cable accommodated in each of the receptacle chambers. With this weight, the probe cable is urged downward and hence can be drawn into the receptacle chamber easily.

In the case where friction induced by contact between the inner surfaces of the receptacle chambers 18a-18d or 18a′-18d′ (shown in FIG. 7) and the probe cables 4 at the time of ingress or egress of the cables poses a problem, the inner surfaces of the receptacle chambers 18a-18d or 18a′-18d′ may be coated so as to diminish the frictional force or a less frictional material may be used as the material of the receptacle housing 15 and that of the partition plates 17a-17c.

Many widely different embodiments of the invention may be configured without departing from the spirit and the scope of the present invention. It should be understood that the present invention is not limited to the specific embodiments described in the specification, except as defined in the appended claims.

1. A probe cable accommodation apparatus comprising a receptacle housing sized to house a plurality of probe cables, the plurality of probe cables each connected at a first end to an ultrasound diagnostic system and at a second end to an ultrasound probe, an interior of the receptacle housing partitioned by a partition plate to form a plurality of receptacle chambers, the probe cables each positioned within a respective receptacle chamber of the plurality of receptacle chambers.

2. The probe cable accommodation apparatus according to claim 1, wherein the receptacle housing includes a plurality of openings that enable the plurality of probe cables to selectively be inserted into and removed from the receptacle chambers such that each probe cable is received in U shape through a respective opening of the plurality of openings into the respective receptacle chamber of the plurality of receptacle chambers.

3. The probe cable accommodation apparatus according to claim 1, further comprising a plurality of rollers each positioned in a respective opening of a plurality of openings for abutment against each of the plurality of probe cables.

4. The probe cable accommodation apparatus according to claim 2, further comprising a plurality of rollers each positioned in a respective opening of the plurality of openings for abutment against each of the plurality of probe cables.

5. The probe cable accommodation apparatus according to claim 3, wherein the plurality of rollers comprises a first roller and a second roller positioned on either side of a respective probe cable, the first and second rollers being rotatable as the probe cable is selectively inserted into and removed from a respective receptacle chamber, the first and second rollers mutually pressing the probe cable.

6. The probe cable accommodation apparatus according to claim 4, wherein the plurality of rollers comprises a first roller and a second roller positioned on either side of a respective probe cable, the first and second rollers being rotatable as the probe cable is selectively inserted into and removed from a respective receptacle chamber, the first and second rollers mutually pressing the probe cable.

7. The probe cable accommodation apparatus according to claim 1, further comprising a cable stopper positioned in each of a plurality of openings to prevent each of the probe cables from being selectively inserted into and removed from the respective receptacle chamber.

8. The probe cable accommodation apparatus according to claim 2, wherein each of the plurality of receptacle chambers is formed in a tubular shape having a parallelogrammic or rhombic section.

9. The probe cable accommodation apparatus according to claim 3, wherein each of the plurality of receptacle chambers is formed in a tubular shape having a parallelogrammic or rhombic section.

10. The probe cable accommodation apparatus according to claim 4, wherein each of the plurality of receptacle chambers is formed in a tubular shape having a parallelogrammic or rhombic section.

11. The probe cable accommodation apparatus according to claim 5, wherein each of the plurality of receptacle chambers is formed in a tubular shape having a parallelogrammic or rhombic section.

12. The probe cable accommodation apparatus according to claim 2, wherein each of the plurality of probe cables is received in U shape through a respective opening toward a bottom of the respective receptacle chamber, said probe cable accommodation apparatus further comprising a movable pulley coupled to a lower end of each of the U-shaped probe cables.

13. The probe cable accommodation apparatus according to claim 12, wherein the movable pulley is urged toward the bottom by a weight.

14. The probe cable accommodation apparatus according to claim 12, wherein the movable pulley is held vertically movably by a rail.

15. The probe cable accommodation apparatus according to claim 12, wherein the movable pulley is supported by an elastic member fixed to the bottom of the associated receptacle chamber.

16. The probe cable accommodation apparatus according to claim 15, wherein the elastic member is a spring.

17. The probe cable accommodation apparatus according to claim 2, wherein each of the plurality of probe cables is received in U shape through a respective opening toward a bottom of the respective receptacle chamber, said probe cable accommodation apparatus further comprising a weight coupled to a lower end of each of the U-shaped probe cables in a slidable state with respect to the probe cable.

18. An ultrasound diagnostic system comprising:
at least one ultrasound probe;
a plurality of probe cables, one of the plurality of probe cables coupled to the at least one ultrasound probe; and
probe cable accommodation apparatus comprising a receptacle housing sized to house a plurality of probe cables, the plurality of probe cables each connected at a first end to an ultrasound diagnostic system and at a second end to an ultrasound probe, an interior of the receptacle housing partitioned by a partition plate to form a plurality of receptacle chambers, the probe cables each positioned within a respective receptacle chamber of the plurality of receptacle chambers.

19. The ultrasound diagnostic system according to claim 18, further comprising:
an operating device configured to receive input of instructions;
an image display device configured to display of an ultrasound image; and
a processing device configured to generate the ultrasound image based on an echo signal obtained by activating the at least one ultrasound probe and to control display of the ultrasound image on the image display device, the operating device being separate from the probe cable accommodation apparatus, the image display device and the processing device and disposed on a side opposite to the processing device with a subject-carrying table positioned therebetween.

20. The ultrasound diagnostic system according to claim 19, further comprising a mounting section for mounting the image display device, and wherein the image display device is mounted to the mounting section through an arm having a movable portion for changing the position of the image display device.
